(19) United States
(12) Patent Application Publication
(10) Pub. No.: US 2003/0082222 A1
(43) Pub. Date: May 1, 2003

(54) Cosmetic particle and method of production thereof

(75) Inventor: Masafumi Miyamoto, Wakayama (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: KAO CORPORATION, Tokyo (JP)

(21) Appl. No.: 10/273,856

(22) Filed: Oct. 21, 2002

(30) Foreign Application Priority Data
Oct. 22, 2001 (JP) 2001-323120

(51) Int. Cl. A61K 7/00; B29B 9/00

(52) U.S. Cl. 424/401; 264/14

(57) ABSTRACT

Provided are a cosmetic particle having a silky feel, a method of producing the cosmetic particle at a low cost and a cosmetic containing the particle. The present invention resides in a method of producing a cosmetic particle having a hardness of 4 or less, the method comprising heating a base material containing wax at a temperature greater than or equal to the melting point of the base material and spraying the base material into a vapor phase at a temperature at which the viscosity of the base material is 600 mPa•s or less, the method of producing a cosmetic particle wherein the base material further contains a silicone compound, a cosmetic particle obtained by these production methods and a cosmetic containing the cosmetic particle.

### FIELD OF THE INVENTION

The present invention relates to a cosmetic particle which can be produced at a low cost and can impart a silky feel by spreading it on the skin, to a method of producing the cosmetic particle and to a cosmetic containing the cosmetic particle.

### PRIOR ART

As additives for imparting a silky feel, inorganic materials such as talc, mica, calcium carbonate and magnesium carbonate and organic materials such as a spherical silicone resin, spherical polyethylene and spherical nylon are widely used. Many of these inorganic materials are inexpensive. However, those having a spherical form are small in number and those having a plate form or a layer and undefined form cannot obtain a silky feel due to rolling which is the feature of spherical particles though can obtain a slippery feel due to layer slipping. Also, because they have a large refractive index, they show the white on the skin. Many of the organic materials are spherical, have a good feel to the touch and have a smaller refractive index than the inorganic materials. Therefore the retained-white problem can be lightened. However, many of these organic materials are obtained by a reaction system such as a suspension polymerization system, dispersion polymerization system and emulsion polymerization system and therefore the process is complicated and the production cost becomes high.

Meanwhile, in the publication of JP-A-57-201248, a method of producing a toner is disclosed, the method comprising spraying a toner material containing 30 mass % or more of polyethylene waxes having a melt viscosity of 10 to 600 mPa•s at 140° C. in a molten state. However, this toner particle has pressure-fixable softness and is therefore not adaptable to a cosmetic particle having a silky feel.

### DISCLOSURE OF THE INVENTION

It is an object of the present invention to provide a cosmetic particle having a silky feel, a method of the production of the cosmetic particle at a low cost and a cosmetic containing the cosmetic particle.

The present invention resides in a method of producing a cosmetic particle having a hardness of 4 or less, comprising heating a base material containing wax and a silicone compound (hereinafter simply referred to as a base material) at a temperature greater than or equal to the melting point of the base material and spraying the base material into a vapor phase at a temperature at which the viscosity of the base material is 600 mPa•s or less.

The cosmetic particle is preferably spherical. The wax is preferably polyethylene wax, polypropylene wax or a mixture of these waxes. The weight average particle diameter of the cosmetic particle is preferably 0.1 to 75 μm.

Further, the present invention provides a cosmetic particle obtained in the above shown method and a cosmetic containing the above particle.

### DETAILED DESCRIPTION OF THE INVENTION

[Method of Producing a Cosmetic Particle]

The wax used in the present invention has a melting point of preferably 80° C. or more, more preferably 120° C. or more, with the view of possessing hardness sufficient to obtain the silky feel required for the cosmetic particle. The upper limit of the temperature is preferably 200° C. or less due to the possibility of thermal decomposition.

Examples of the wax include synthetic waxes such as higher fatty acid esters, hydrocarbon waxes (e.g., paraffin wax, microcrystalline wax, polyethylene wax, polypropylene wax and ethylene/propylene copolymer wax); and natural waxes such as carnauba wax. Among these waxes, polyethylene wax, polypropylene wax, ethylene/propylene copolymer wax and mixtures of these waxes are preferable and polyethylene wax, polypropylene wax and mixtures of these waxes are more preferable.

Although these polyethylene wax, polypropylene wax and ethylene/propylene copolymer wax may be either straight-chain types or branched chain types, straight-chain types are preferable to provide hardness imparting a silky feel. The molecular weight is preferably 500 to 20,000 and more preferably 1,000 to 10,000 to make the viscosity 600 mPa•s or less when spraying. Here, the molecular weight can be found by a viscosity method or a steam osmotic pressure method.

The content of the wax in the base material used in the present invention is preferably 30 mass % or more, more preferably 60 mass % or more, and particularly preferably 80 mass % or more. The upper limit is preferably 99.9 mass % or less, more preferably 99 mass % or less.

The melting point of the base material in the present invention is preferably 80° C. or more and more preferably 120° C. or more with the view of obtaining hardness sufficient to obtain the silky feel required for the cosmetic particle. The upper limit of temperataure is preferably 200° C. or less due to the possibility of thermal decomposition.

Moreover, the base material of the present invention contains a silicone compound as a spray-improving adjuvant for decreasing the particle diameter of a liquid droplet when spraying. Incorporation of the silicone compound makes the surface of the particle more hydrophobic and more preferable to cosmetic.

The silicone compound means a solid or liquid material having a siloxane skeleton as its molecular structure. Specific examples of the silicone compound include cyclic silicones such as octylmethylcyclotetrasiloxane, decamethylcyclopentasiloxane and dodecamethylcyclohexasiloxane, chain silicones such as dimethylpolysiloxane and methylphenylpolysiloxane, amino modified silicones, polyether modified silicones, methylphenylpolysiloxanes, fatty acid modified silicones, alcohol(carbinol) modified silicones, alkoxy modified silicones, epoxy modified silicones, fluorine modified silicones and alkyl modified silicones. The weight average molecular weight of thesilicone compound is 1000 to 500,000, more preferably 1000 to 200,000 by GPC(gel permeation chromatography) using polystylene as standard and chloroform as eluent.

Among these silicone compounds, alkyl-modified silicones, alkoxy-modified silicones or alcohol(carbinol)-modified silicones containing an alkyl, alkoxy or hydroxyalkyl group being either straight or branched, preferably having 8 to 100 carbon atoms, more preferably 8 to 50, much more preferably 8 to 22, are preferable because of a good compatibility with wax. These silicones may be either chain types or cyclic types. The substituent, such as an alkyl, an alkoxy or a hydroxyalky group, may be attached to the side chain, at one end and/or both ends, of the polysiloxane. Examples of the substitutents are analkyl group such as octyl, 2-ethylhexyl, decyl, myristyl, cetyl, stearyl or behenyl, a hydroxyalkyl group of the above shown alkyl group, having a hydroxide group, or an alkoxy group of the above shown alkyl group, having an oxygen atom.

The content of the silicone compound in the base material according to the present invention is preferably 0.1 mass % or more, more preferably 1 mass % or more, and preferably 20 mass % or less, more preferably 10 mass % or less, in the base material with the view of obtaining high particle hardness, being free from deformation even under pressure and obtaining good spreading ability and a silky feel.

The base material of the present invention may contain, besides the aforementioned components, for example, organic or inorganic solid or liquid components for the purposes of raising hardness, lowering viscosity and surface tension and imparting the functions required for the cosmetic particle. For example, vegetable oil and fats such as squalene and palm oil, oils such as liquid paraffin, vaseline, lanolin, ceresin, higher fatty acids and higher alcohols, oil soluble polymers, colorants such as inorganic and organic pigments and organic dyes, surfactants such as anionic surfactants, cationic surfactants and nonionic surfactants, antiseptics, antioxidants, perfumes, ultraviolet absorbers, humectants, blood circulation promoters, cold-feel imparting agents, antiperspirant agents, bactericidal agents and skin protecting agents may be compounded appropriately to the extent that the effect of the present invention is not impaired.

In order to obtain the cosmeticparticle of the present invention, first the base material is melted under heating at a temperature greater than or equal to the melting point of the base material and a silicone compound is melted according to the need when heating. In addition, the organic or inorganic solid or liquid component described above may be melted or dispersed.

Next, this molten material is sprayed into a vapor phase kept at preferably 5 to 50° C. by using a rotary disk atomizer and a one-fluid or two- or more-fluid nozzle to recover the cooled and solidified particles. Preferably, the molten material is sprayed into a vapor phase by using a two- or more-fluid nozzle together with compressed gas. As the compressed gas used as the fluid, compressed air or nitrogen having a pressure of preferably 9.8×10⁴ Pa or more and more preferably 9.8 to 29.4×10⁴ Pa may be used. As the gas, one heated to the spray temperature is preferably used because clogging due to cooling at the nozzle portion can be prevented and particles can be produced continuously. The spray temperature may be a temperature at which the viscosity of the base material becomes 600 mPa•s or less, preferably 300 mPa•s or less and more preferably 100 mPa•s or less. The viscosity is preferably 10 mPa•s or more though there is no particular limitation to its lower temperature. If the viscosity of the base material is 600 mPa•s or less when spraying, good spraying ability is obtained and a particle diameter preferable as the cosmetic particle is obtained.

[Cosmetic Particle]

The shape of the cosmetic particle of the present invention is, although it is not particularly limited, preferably spherical for use in cosmetic applications because such a spherical particle is free from a gritty feel. Also, the weight average particle diameter of the particle is preferably 0.1 to 75 μm, more preferably 5 to 40 μm and particularly preferably 5 to 20 μm from the view point of imparting a good feel to the skin as a cosmetic. Here, the weight average particle diameter is a value measured in ethanol by using an LS-230 (manufactured by COULTER).

The hardness of the cosmetic particle of the present invention is 4 or less and preferably 2 or less as a value measured by a measuring method according to JIS K2207. Although no particular limitation to the upper limit of the hardness, the hardness is preferably a value exceeding 0 from the condition of measurement. When the hardness is in a range smaller than or equal to 4, the cosmetic particle is reformed by external pressure with difficulty and has high spreading ability and a good silky feel.

In the present invention, particularly a particle obtained by melting under heating the base material containing the wax and the silicone compound at a temperature greater than or equal to the melting point of the base material and by spraying the base material into a vapor phase at a temperature at which the viscosity of the base material becomes 600 mPa•s or less is preferable because the silicone compound is present on the surface of the particle and the silky feel of the particle is therefore improved.

[Cosmetic]

The content of the particle according to the present invention in the cosmetic of the present invention is preferably 0.1 to 50 mass % and particularly preferably 1 to 30 mass % though it may be optionally selected corresponding to the purpose of the cosmetic without any particular limitation.

No particular limitation is imposed on the state of the cosmetic of the present invention and the cosmetic may be any one of a water-in-oil type or oil-in-water type emulsion cosmetic, oily cosmetic, spray cosmetic, stick cosmetic, aqueous cosmetic, sheet cosmetic and gel cosmetic. Also, there is no particular limitation to the type of cosmetic of the present invention. Examples of the type include skin cosmetics such as a pack, foundation, lipstick, lotion, cold cream, hand cream, skin detergent, softener cosmetic, nutrient cosmetic, astringent cosmetic, whitening beauty cosmetic, wrinkle-improving cosmetic, aging preventive cosmetic, washing cosmetic, antiperspirant agents and deodorant; and hair cosmetics such as a shampoo, rinse, treatment, hairdressing and hair tonic.

The cosmetic of the present invention preferably contains alcohol. Examples of the alcohol include monohydric or polyhydric alcohols having 1 to 6 carbon atoms such as ethanol, glycerol, 1,3-butylene glycol, propylene glycol and sorbitol. Among these alcohols, a monovalent alcohol, particularly, ethanol is preferable. The amount of the alcohol to be compounded is preferably 2 to 30 mass %, preferably 5 to 30 mass %, more preferably 5 to 20 mass %, in the cosmetic of the present invention. Alternatively it is particularly preferably 1 to 50 times by mass, more preferably 2 to 50 times by mass, as much as the cosmetic particle according to the present invention.

Other components used generally as cosmetic components may be further compounded in the cosmetic of the present invention as required corresponding to the state, type, and the like of the above cosmetic to the extent that the effect of the present invention is not impaired.

Examples of these cosmetic components include extender pigments such as mica, talc, sericite, kaolin, nylon powder, polymethylsilsesquioxane and barium sulfate; inorganic pigments such as titanium oxide, zinc oxide and iron oxide; powders prepared by processing the above powders by surface hydrophobic treatment such as silicone treatment, metal soap treatment or N-acylglutamic acid treatment; hydrocarbons such as solid or liquid paraffin, microcrystalline wax, vaseline, ceresin, ozokerite and montan wax; vegetable oil and fats, animal oil and fats or waxes such as olive, mineral wax, carnauba wax, lanolin and spermaceti wax; fatty acids or their esters such as stearic acid, palmitic acid, oleic acid, glycerol monostearate, glycerol distearate, glycerol monooleate, isopropyl myristate, isopropyl stearate and butyl stearate; higher alcohols such as cetyl alcohol, stearyl alcohol, palmityl alcohol and hexyldodecyl alcohol; adsorbing or thickening agents such as cationized celluloses, carboxybetaine type polymers and cationized silicone; polyhydric alcohols having moisture retentive ability such as glycol and sorbitol; pharmaceutically effective components such as whitening beauty cosmetics, analgesic and antiphlogistic agents, antipruritic agents, bactericidal and disinfectant agents, astringent agents, skin softeners and hormonal agents; water; surfactants; W/O or O/W type emulsifying agents; emulsifying agents for silicone oil such as polyether modified silicone, polyether alkyl modified silicone and glyceryl ether modified silicone; thickening agents such as methyl cellulose, ethyl cellulose, carboxymethyl cellulose, polyacrylic acids, traganth, agar and gelatin; and besides, emulsion stabilizers, chelating agents, ultraviolet protectors, pH regulators, antiseptics, dyes and perfumes.

The cosmetic particle of the present invention can be produced at a low cost, has high spreading ability on the skin and can impart a silky feel to the skin.

### EXAMPLES

In the following examples, viscosities when splaying are values measured using a B-type viscometer at 60 rpm, for one minute at the spray temperature (as the rotor, a type having a measuring scale ranging from 5 to 95 is used). The hardness of the particle is a value obtained by molding particle to plate and measuring according to JIS K2207. The melting point of the base material is a value measured according to JIS K0064:1992.

### Example 1

As the wax, 100 mass parts of polyethylene wax, Mitsui High Wax HW-200P, manufactured by Mitsui Chemical, having a molecular weight of 2000 and a melting point of 122° C., was used. As the silicone compound, 5 mass parts of D5 (decamethylcyclopentanesiloxane), SH245, manufactured by Dow Corning Toray Silicone CO., Ltd., was added to obtain a mixture having a melting point of 122° C. The mixture was then melted under heating at 180° C. and cooled by spraying it into a 25° C. vapor phase together with a 180° C. nitrogen stream by using a two-fluid nozzle (Glass Atomizer M-model, manufactured by SANSYO) to recover it as a solid particle. The viscosity of the base material when it was sprayed was 58 mPa•s. The hardness of the resulting cosmetic particle was 1.5.

### Example 2

The same procedures as in Example 1 were conducted except that as the silicone compound, alkyl modified silicone (KF413, manufactured by Shin-Etsu Silicones (Co., Ltd.)) was added in an amount of 5 mass parts to 100 mass parts of polyethylene wax (melting point of the mixture: 122° C.) in Example 1, to obtain a solid particle. The viscosity of the base material when it was sprayed was 58 mPa•s. The hardness of the resulting cosmetic particle was 1.

### Comparative Example 1

The same procedures as in Example 1 were conducted except that as the wax, polyethylene wax (Mitsui High Wax HW-400P, manufactured by Mitsui Chemical (Co., Ltd.), molecular weight: 4000 and melting point 126° C.) was used in Example 1, to obtain a solid particle. The viscosity of the base material when it was sprayed was 650 mPa•s. The hardness of the resulting cosmetic particle was 1 or less.

### Comparative Example 2

The same procedures as in Example 1 were conducted except that behenic acid (melting point: 81° C.) was used in place of the wax in Example 1, to obtain a solid particle. The viscosity of the base material when it was sprayed was 10 mPa•s. The hardness of the resulting cosmetic particle was 10 or more.

### Test Example

The weight average particle diameter of each of the cosmetic particles obtained in Examples 1 to 4 and Comparative Examples 1 and 2 was measured using an LS-230 (manufactured by COULTER) in ethanol. Also, the spreading ability and feel to the touch as a cosmetic were evaluated using the following method. The results are shown in Table 1.

<Spreading Ability>

Plural particles were put between the fingers of an expert panelist (one member) and these fingers were rubbed against each other to evaluate the diffused state of the particles between these fingers according to the following standard.

◯: Spread uniformly.

Δ: Spread ununiformly.

X: The particles were crushed and not spread.

<Feel to the Touch>

An expert panelist (one member) functionally evaluated each particle as a cosmetic.

TABLE 1

| | Weight average | | |
|---|---|---|---|
| | particle diameter | Spreading | |
| | (μm) | ability | Feel to touch |
| Example 1 | 20 | ∘ | Very silky feel |
| Example 2 | 13 | ∘ | Very silky feel |
| Comparative | 85 | Δ | Gritten feel |
| example 1 | | | |
| Comparative | 20 | x (crushed) | Wax-like slimy feel |
| example 2 | | | |

As is clear from Table 1, the cosmetic particle of the present invention was superior in spreading ability on the skin and feel to the touch as a cosmetic.

### Example 3

### Hand Cream

The following (1) to (8) were mixed under heating at 73° C. and the following (9) to (12) were mixed under heating at 75° C. The both were emulsified uniformly using a homomixer, followed by defoaming and cooling to obtain a hand cream. This hand cream had good spreading ability on the skin and a silky feel.

<Composition of the hand cream>

| | | | |
|---|---|---|---|
| (1) | Stearic acid | | 8 |
| | | | mass % |
| (2) | Cetyl palmitate | | 2 |
| (3) | Cetanol | | 3 |
| (4) | Lanolin | | 1 |
| (5) | Liquid paraffin (light) | | 15 |
| (6) | Decamethylcyclopentanesiloxane | | 1 |
| (7) | Polyoxyethylene (20) sorbitan | | 1 |
| | monostearate (EO average mole number | | |
| | of 20) | | |
| (8) | Particle obtained in Example 1 | | 5 |
| (9) | Triethanolamine | | 1 |
| (10) | Propylene glycol | | 5 |
| (11) | Sorbitol (70%) | | 2 |
| (12) | Purified water | the balance for | 100 |

1. A method of producing a cosmetic particle having a hardness of 4 or less, comprising heating a base material containing wax and a silicone compound (hereinafter simply referred to as a base material) at a temperature greater than or equal to the melting point of the base material and spraying the base material into a vapor phase at a temperature at which the viscosity of the base material is 600 mPa•s or less.

2. A method according to claim 1, wherein the cosmetic particle is spherical.

3. A method according to claim 1 or 2, wherein the wax is selected from the group consisting of polyethylene wax, polypropylene wax and a mixture of these waxes.

4. A method according to claim 1 or 2, wherein the weight-average particle diameter of the cosmetic particle is 0.1 to 75 μm.

5. A method according to claim 1, wherein the silicone compound is an alkyl-modified, alkoxy-modified or alcohol(carbinol)-modified silicone.

6. A cosmetic particle obtained by the method as claimed in claim 1 or 2.

7. A cosmetic containing the particle as claimed in claim 6.
